(19) United States
(12) Patent Application Publication
(10) Pub. No.: US 2026/0272708 A1
(43) Pub. Date: Sep. 17, 2026

(54) IMPLANT AND ARRANGEMENT COMPRISING A RADIATION SOURCE AND AN IMPLANT

(71) Applicant: RHEINISCH-WESTFÄLISCHE TECHNISCHE HOCHSCHULE (RWTH) AACHEN, Aachen (DE)

(72) Inventors: Benedict BAUER, Aachen (DE); Thomas Gerhard GRIES, Aachen (DE); Ioana SLABU; Thomas SCHMITZ-RODE, Aachen (DE)

(73) Assignee: RHEINISCH-WESTFÄLISCHE TECHNISCHE HOCHSCHULE (RWTH) AACHEN, Aachen (DE)

(21) Appl. No.: 18/875,702

(22) PCT Filed: Jun. 14, 2023
(86) PCT No.: PCT/EP2023/065995

§ 371 Date: Dec. 16, 2024

(30) Foreign Application Priority Data
Jun. 21, 2022 (DE) 10 2022 115 394.0

(51) Int. Cl. A61F 7/12; A61F 2/90; A61F 7/00; A61L 31/12; A61L 31/14; A61L 31/16; A61N 2/02

(52) U.S. Cl. CPC A61F 7/12; A61F 2/90; A61L 31/129; A61L 31/148; A61L 31/16; A61N 2/02; A61F 2007/009; A61F 2007/126; A61F 2210/0076

(57) ABSTRACT

The present invention relates to an implant for implanting in a body, in particular in a hollow organ or a vessel of a body, wherein the implant is constructed from a filament containing at least one polymer matrix material in which a magnetically heatable filler is arranged, wherein the filament has a cross section with a core-sheath structure, wherein the core comprises a first in particular polymer matrix material having a first melting point and wherein the sheath comprises a second polymer matrix material having a second melting point, wherein the magnetically heatable filler is present at least in the second matrix material, wherein the second melting point is lower than the first melting point, wherein the second melting point is in a range from ≥45° C. in particular to ≤100° C.

This application is a national phase of International Application No. PCT/EP2023/065995 filed Jun. 14, 2023, which claims priority to German Application No. 102022115394.0 filed Jun. 21, 2022, each of which is hereby incorporated herein by reference in its entirety.

The present invention relates to an implant, such as a stent in particular. The present invention relates in particular to an implant which is preferably insertable into a hollow organ or a vessel of a body and which has particularly preferred properties. The present invention furthermore relates to an arrangement comprising such an implant and a radiation source for emitting electromagnetic radiation.

There has been research into biodegradable polymer stents for quite some time now since-unlike conventional metal stents made of nitinol, for instance-they remain in the body only temporarily. This is because the stent is only required for a short period of time in most applications. Permanent retention is linked to disadvantages, such as the risk of in-stent restenosis, neointimal hyperplasia and incomplete healing. It is very difficult to remove metallic stents again, and they represent a problem in the case of restenosis. Biodegradable polymer stents offer great potential but have seen limited use to date on account of their low material-induced radial strength.

EP 3 277 375 B1 describes that iron/platinum (Fe/Pt) particles can be dispersed within a polymer and applied in or on medical equipment or connected directly to the latter and magnetized. The magnetized devices are used to attract and capture magnetically marked cells and/or keep the latter in vivo on the surface of the device. Magnetic devices are particularly useful for capturing and securing cells exposed to the loads and forces of biological fluid flow, e.g. for capturing and securing these cells to stents implanted post angioplasty, in order to form a patented endothelial surface that prevents the occurrence of restenosis. They can also be used to improve tissue integration at the implantation site of a prosthesis, e.g. a hip or knee prosthesis with a metal surface, or to reduce bone erosion around a bone screw, a pin or a plate made of metal. The devices can be produced from a metal, a polymer or a combination thereof.

EP 2 249 804 B1 describes nanoparticle-containing implantable and biologically degradable medical products and the use thereof for thermal therapeutic aftercare following surgical removal of tumours and cancerous ulcers. Such a medical product is available as tissue, sponge or film, in particular, in order to be flexible or deformable, with the medical product containing magnetic particles which can be excited to generate heat by way of an alternating magnetic field and thereby heat the medical product.

US 2008/0071353 A1 describes stents containing magnetic induction nanoparticles, wherein the particles have a metallic coating.

Such solutions known from the prior art may still have potential for improvement, especially in view of improved applicability.

Therefore, the problem to be solved by the present invention is that of developing a measure which at least partly overcomes at least one disadvantage of the prior art. In particular, a problem to be solved by the present invention is that of developing a measure with which the applicability of an implant, in particular of a stent, can be improved.

According to the invention, the problem is solved by an implant having the features of claim 1. According to the invention, the problem is also solved by an arrangement having the features of claim 13. Preferred configurations of the invention are disclosed in the dependent claims, in the description and in the figures, wherein further features described or shown in the dependent claims or in the description or the figures may represent, either on their own or in any desired combination, subject matter of the invention unless the contrary clearly emerges from the context.

The present invention relates to an implant for implanting in a body, in particular in a hollow organ or a vessel of a body, wherein the implant is constructed from a filament containing at least one polymer matrix material in which a magnetically heatable filler is arranged, wherein the filament has a cross section with a core-sheath structure, wherein the core comprises a first, in particular polymer, matrix material having a first melting point and wherein the sheath comprises a second, polymer matrix material having a second melting point, wherein the magnetically heatable filler is present at least in the second matrix material, wherein the second melting point is lower than the first melting point, wherein the second melting point is in a range from ≥45° C. in particular to ≤100° C.

Such an implant has clear advantages over solutions from the prior art, especially in relation to a combination of being easily introducible into a hollow organ or a vessel of a body in conjunction with good mechanical properties, especially a great radial strength following the introduction in the hollow organ or vessel.

The implant described here serves in particular for implantation in a body of a living being in particular, for example in a human body, with implantation in a vessel or hollow organ of the body, for example in an artery, the windpipe, bile ducts and ureter and urethra, being particularly preferred. To this end, the implant may have in particular a certain amount of flexibility, for instance in order to be introduced into the body, for instance into the vessel or hollow organ, in a compressed and/or deformed state and in order to adopt its desired application shape or application geometry at the desired position.

Moreover, the implant is in particular of a type that only remains temporarily within the body and in this context is constructed at least in part, particularly preferably in full, from biologically degradable substances, i.e. the first and the second matrix material are preferably biodegradable. A biodegradable plastic is understood to be one conforming to the basic understanding, for example pursuant to ASTM F2902-16.

The implant is formed from a filament, which can also be referred to as a fibre. To produce the implant, the filament can be processed in a manner known per se using fibre processing methods known to a person skilled in the art. Examples of such fibre processing methods or textile further-processing processes include the crossing or looping of the filament, as occurs during weaving, knitting by loop forming, knitting by loop drawing, lace production, braiding and the production of tufted products. Furthermore, the implant can be a nonwoven, with it however possibly being preferable for the filament to precisely not be a nonwoven.

The filament may have been created in advance by a spinning process. To this end, coextrusion can be used to create the core-sheath structure. In principle, a two-layer structure in particular can be present as core-sheath structure.

The creation of a core-sheath structure at the level of fibres is rendered possible by a coextrusion method in particular, without being restricted thereto. It is preferable for the core to have a thread-shaped configuration and for the sheath to have a tubular structure and at least partially envelop the core. In other words, the filament from which the implant is shaped by a fibre processing method in particular is embodied as a multi-layer structure at the level of fibres. The core is thread-shaped and hence formed from solid material. The envelope is tubular and embodied around the core. For example, this is rendered possible by using two coaxial nozzles in an extrusion process, the nozzles forming the core in the interior and the sheath, as a tubular structure, radially around the core.

Within the meaning of the present invention, tubular structure should in particular be understood to mean that the sheath has a tubular structure, and so the sheath covers the core on the outside, i.e. radially, especially completely or in full, by virtue of the sheath extending around the core. In that case, the interior of the tubular form is filled, in particular completely filled, by the core. In particular, both the core and the sheath can form preferably a closed layer, with the layers preferably being pore-free. However, pores in the core or in the sheath should in principle be encompassed by the present invention.

Particularly high strength can be achieved by a coextrusion method for the production of the filament since the materials, especially polymer materials of sheath and core, are oriented. For example, this is an advantage over an additive manufacturing method, in which the materials are usually unoriented.

The filament contains at least one polymer matrix material in which a magnetically heatable filler is arranged. In particular, the magnetically heatable filler can be present in the matrix material in homogeneously finely distributed fashion, wherein, in the case of the implant described herein, provision can be made for the filler to be present only in a predefined region along the cross section of the filament, as is described in greater detail below. A more homogeneous distribution of the filler ultimately makes heating possible in more homogeneous and defined fashion, as described below.

As regards the magnetically heatable filler, the latter should be understood within the meaning of the present invention as one that heats up when triggered by a magnetic field or an electromagnetic field. In particular, heating is implemented in defined and reproducible fashion such that the effect of applying a magnetic field with known parameters leads to the filler or, in particular, the filament being heatable to a defined temperature value. It can be particularly advantageous if the filler is heatable in such a way that the filament has a temperature in a range from 40° C. to 100° C., preferably in a range from 45° C. to 100° C., as described below.

In more detail, the core comprises a first in particular polymer matrix material having a first melting point, and the sheath comprises a second polymer matrix material having a second melting point. Accordingly, in the case of the implant described here, provision is made for core and sheath to be embodied from different materials that differ in terms of their melting point.

In particular, the melting point is determined at 1.013 bar.

As regards the melting points, provision is made for, in more detail, the second melting point, i.e. the melting point of the matrix material of the envelope, to be lower than the first melting point, i.e. the melting point of the matrix material of the core, wherein the second melting point is in a range from ≥45° C. in particular to ≤100° C. Here, a melting point difference between the first melting point and the second melting point that is in a range of ≥5° C., for example ≥20° C., is preferably provided.

As regards the arrangement of the magnetically heatable filler, provision is furthermore made for the latter to be present at least in the second matrix material, i.e., in other words, in the envelope. In particular, this enables a defined heatability of the envelope or second matrix material, and so heating is implemented locally directly in the component that should be melted or at least partially melted, as described below. By preference, the filler can be present predominantly in the outer region, whereas the core contains less of the magnetically heatable filler, or, in particular, is free from the latter. Hence, it is preferable as a matter of principle for the filler to be loaded more in the sheath than in the core. A polymer reinforcement structure is formed in the core as a result. Within the meaning of the present invention, reinforcement structure of the core should in particular be understood to mean that the core represents a reinforcement for the sheath, in particular by virtue of the core having a greater stability or strength than the sheath. In particular, the reinforcement can relate to the tensile strength of the filament such that a very stable implant is present, despite the meltability of the envelope.

The above-described configuration provides an option of increasing the radial force for braided stent implants without a reduction in flexibility. This is based on the implant or the stent being defined in such a way with respect to its specific material selection that sparing heating of the implant, in particular of the envelope of the implant, allows crossing points or nodes of the implant structure to be connected to one another after the implant has been introduced into the body. In more detail, it is possible to integrally bond crossing points of the braided stent postoperatively and noninvasively by brief electromagnetic heating. This heating is imparted by the filler, or preferably the magnetically heatable nanoparticles (MNP), by means of which the stent can be heated locally and in very controlled fashion to a specific temperature level.

Accordingly, within the meaning of the present invention, nodes should be understood to mean regions or points of different strands that are in direct vicinity of one another, in particular in contact with one another, and crossing points should be understood to mean those regions or points in which the previously adjacently present strands or nodes are integrally bonded to one another.

To this end, a low-temperature-melting material component is used in the envelope as defined above, said material component being melted locally, and optionally under contact pressure from a balloon catheter, by appropriate heating and hence preventing the fibres from slipping under a radial load. As a result of the difference between the melting points of the materials of the envelope and of the core which sets in here, it is possible to leave the structure of the core intact such that the basic stability is not reduced and such that, further, the three-dimensional structure of the implant is not modified as a result of the envelope melting.

This type of postoperative connection of the crossing points is advantageous since the stent must be present in compressed form in a catheter for minimally invasive release, and crossing points already connected at this stage would reduce the flexibility of the stent. Hence, the configuration of the implant can bring about a postoperative increase in stability, in particular in respect of a radial strength, wherein sufficient flexibility for the introduction of the implant is nevertheless present.

The invention thus solves the problem of an insufficient radial strength of polymer stents. This decisively increases the use spectrum of these stents. Metallic stents, which are currently preferably used and have disadvantages in particular as a result of permanent retention within the body, could be at least partially replaced thereby. This advantage is not to the detriment of the compressibility of the stent since the precisely stent struts can still slide off one another under load during the introduction and can furthermore provide the option of a minimally invasive intervention.

Further advantages of the provision of the filler in the sheath can thus be seen in that the orientability of the core component is not disturbed by the filler or nanoparticles and further that the success as regards the connection of crossing points is verifiable by way of imaging methods, for example by means of magnetic resonance imaging (MRI) and/or magnetic particle imaging (MPI). This can therefore be advantageous, in particular, since the crossing points are connected postoperatively, as described above, and hence, on a basic level, it is not easily ascertainable whether the crossing point connection was successful.

So as not to damage healthy, surrounding tissue, the melting point of the second matrix material, i.e. the second melting point, can be restricted to a temperature range that prevents damage to the surrounding tissue.

In principle, the second melting point can be in a range from ≥45° C. in particular to ≤100° C., for example from ≥45° C. to ≤75° C. This ensures that the structure of the implant remains intact, even in the event of an elevated body temperature, but that no tissue damage need be feared, at least in the event of a short-term temperature exposure.

In one embodiment, the second melting point can be in a range from ≥45° C. to ≤65° C. This configuration, in particular, can allow very sparing connecting of the crossing points since the influence of the temperature on the surrounding tissue is very low and temperatures up to 65° C. do not yet cause tissue damage, or this can be reliably prevented, in the event of the exposure durations applicable in this case.

A thermoablation method can additionally be carried out, especially if the second melting point, i.e. the melting point of the second matrix material, is in a range above 50° C., for example in a range from >65° C. to ≤100° C., in addition to the connection of nodes, the latter being realizable at a temperature above the aforementioned melting point. To this end, the particles or the matrix material of the sheath can be heated to a temperature in a range from 41° C. to 44° C., for example, without this being a risk to the stability of the implant. In this context, it can be particularly advantageous that, according to the invention, it is also possible to set very narrow temperature ranges in very defined fashion, for instance in a range from 41° C. to 44° C., or else other temperature ranges located in particular within the aforementioned ranges. In particular, this is possible by way of the selection and loading of the fillers and the parameters of the magnetic excitation thereof.

Performance of thermoablation methods could be allowed in the aforementioned temperature ranges, in a manner analogous to high-intensive focused ultrasound (HIFU), radiofrequency-induced thermotherapy (RFITT) or laser-induced interstitial thermotherapy (LITT). Thermoablation methods are directed to devitalization (coagulation) of the target tissue, for instance of tumour tissue, by way of apoptotic cell damage in particular using temperatures from 41° C. to 44° C., wherein unwanted necrosis can be avoided. Within the scope of thermoablation methods, especially in the lower temperature range, the described implant can in principle serve for the creation of therapeutically effective heat, in particular, or for imaging purposes.

As regards the first matrix material, i.e. the matrix material of the core, it can be preferable for this to be selected from the group consisting of polylactides (PLA), polylactide-co-glycolide (PLGA), polyglycolides (PGA), poly-4-hydroxybutyrates (P4HB), polydioxanone. In an alternative to that or in addition, provision can be made for the second matrix material, i.e. the matrix material of the envelope, to be formed from the group consisting of polycaprolactone (PCL) and a copolymer of polyglycolic acid and ¿-caprolactone, for instance composed of 75 wt % polyglycolic acid and 25 wt %-caprolactone (PGCL). It was found that these materials in particular can be advantageously used within the scope of the present invention since they form appropriate stabilities and furthermore are within the predefined temperature ranges of the melting points. Moreover, these materials are biodegradable, and so the formation of biodegradable stents is possible. Finally, these materials can preferably be processed by coextrusion, the latter being a preferred production method for the described implant.

It can furthermore be preferable for an active ingredient to be present in the second matrix material. The scope of application of the implant can be further increased in this configuration. As a matter of principle, the active ingredient, which for example can be released in retarded fashion in a manner known per se, is freely selectable, and the amount of the active ingredient in the sheath can depend as a matter of principle on the desired application.

By preference, the magnetically heatable filler can comprise a ferromagnetic or ferrimagnetic, in particular superparamagnetic, filler. The superparamagnetic effect for instance of nanoferrites describes a magnetic property of very small particles of a ferromagnetic or ferrimagnetic material. The superparamagnetic effect refers to no permanent magnetization of said material even at temperatures below the Curie temperature once a previously applied magnetic field has been deactivated. An accumulation of nanoferrites in the polymer matrix therefore behaves macroscopically like a paramagnet, but nevertheless has a high magnetic saturation of a ferromagnet and accordingly reacts like a soft-magnetic ferromagnet to inductive fields. In contrast to a paramagnet, it is not individual atoms but small magnetic particles that change their magnetization direction independently of one another. Thus, such superparamagnetic substances, in particular superparamagnetic nanoferrite particles with an adjustable saturation temperature, are preferably used to control the local heating.

An advantage of such fillers can in particular be found in the fact that these enable appropriate heating of the structure with an adjustable saturation temperature in particularly defined fashion and/or within a short period of time. Therefore, heating of the matrix material and thus forming of linkage points by melting the matrix material using an implant according to the invention can be performed particularly sparingly.

Examples of such superparamagnetic fillers comprise ferrites in particular, for example superparamagnetic iron oxide particles, for instance magnetite or maghemite.

In particular, it can be advantageous if the filler, in particular the superparamagnetic filler, has a crystallite size, which can also be referred to as core size or magnetic core size, in a range from greater than or equal to 3 nm to less than or equal to 100 nm, for instance greater than or equal to 10 nm to less than or equal to 30 nm, wherein the crystallite size, at which a material has superparamagnetic properties, can be very material-dependent. Such nanoparticles, also referred to as magnetic nanoparticles (MNP), can particularly effectively enable the described advantages in diagnostic (contrast agent in magnetic resonance imaging (MRI)) and therapeutic applications, but also when melting the sheath, on account of their physical properties.

This is because nanoparticles, on account of their magnetic attraction and the large surface-to-volume ratio, tend to form agglomerates with dimensions of a few micrometres (macroscopic agglomerates), for example of ≤10 μm. The formation of agglomerates in the production process can only be influenced to a certain extent or with much outlay when producing nanocomposites. The agglomerates act like defects and decisively influence the properties of the resultant nanocomposites. One option for improving the material properties lies in a homogeneous distribution of the particles in the end product. Two production methods, the melt mixing process and solution mixing process, are currently applied in industry for the production of nanocomposites. In-situ polymerization, particle functionalization or ultrasonic waves are used to homogenize the nanoparticles in the matrix.

The spinning process in particular is advantageous; therein, a second component, the core, is also spun out in addition to the particle-loaded functional component, the sheath, in order to produce the implant according to the invention, the second component making it possible to ensure a significant improvement in the mechanical strength both during the spinning process and following the completion. Hence, coextrusion of two materials, in particular, is used in the melt spinning process.

The spinning process follows the melt mixing process, for example with the twin-screw extruder. This can be implemented in one step, but also in two steps.

The product of the melt mixing process, which is also referred to as compounding, is granular material. The latter is then subsequently spun out into fibres in a melt spinning process.

Furthermore, it can be preferable for the implant to have a tubular structure, i.e. a pipe-shaped or tubing-shaped structure in particular. For example, the implant can be a stent. Hence, the filament constructed from a core-sheath structure can be processed to form the tubular structure by way of fibre processing processes.

Further advantageously, the filament can have a crossed or a looped structure. Hence, the filament can be processed into the implant in particular using fibre processing processes known per se, and in particular need not be a nonwoven in this case. This allows creation of a defined and equally stable structure, which can improve the use as an implant.

In particular, it can be preferable for the filament to have a braided structure. A braided structure, in particular, can have advantages for therapy in a hollow organ or in a vessel. In particular, this can render possible a textile stent with a braided structure, which has great flexibility and experiences little mechanical load on the fibres during the production. Moreover, a braid in particular can be compressed without problem for the purpose of being introduced into a hollow organ or a vessel, and can in this case obtain its desired non-compressed shape within the hollow organ on account of a sufficient mechanical restoring force. Hence, a braid can have advantages over other products made by fibre-processing methods or else over nonwovens or nonwoven fabrics. Furthermore, the radial strength can be further improved by the linkage points, as described above.

Furthermore, it can be preferable for the magnetically heatable filler to be present in the sheath in a proportion of greater than or equal to 0.1 wt % to less than or equal to 90 wt %, preferably greater than or equal to 3 wt % to less than or equal to 30 wt %. In this embodiment, in particular, the filler can be heated in such a way that the implant is heated to the above-described desired temperature. In this case, the implant according to the invention especially in this configuration may have reduced mechanical properties, such as a reduced stability, in the case of a mono-component structure, i.e. without the reinforcement layer, and so the present invention can have effective advantages especially in this configuration.

As regards further technical features and advantages of the implant, reference is made to the description of the arrangement, to the figures and the description of the figures, and vice versa.

An arrangement of a radiation source for emitting electromagnetic radiation and an implant is also described, wherein the implant contains a magnetically heatable filler. The arrangement is characterized in that the implant is configured as described above.

By way of the radiation source, such an arrangement allows the magnetically heatable filler to be inductively heated in defined and reproducible fashion by magnetic relaxation processes, and the implant can thus enable linkage points in a defined manner by melting and enabling an integral bond to form at different points of the implant.

To this end, it can be advantageous, especially for use in the body, for the implant and the radiation source to be matched to one another in such a way that the implant is heatable, at least in the sheath, to a temperature in a range from ≥45° C. to ≤100° C. by electromagnetic radiation emitted by the radiation source. For example, this can enable an effective and equally sparing formation of linkage points or crossing points and optionally cancerous tissue destruction.

Corresponding matching of emitted radiation to the implant can be made possible on part of the electromagnetic radiation, especially by setting a suitable frequency. Exemplary frequencies and field amplitudes comprise a range from 10 kHz to 1 MHz and 1 kA/m to 100 kA/m.

Such ranges can be advantageous since a suitable combination of frequency and field amplitude can particularly effectively counteract inadvertent heating of tissue as a result of the formation of so-called eddy currents. In particular, consideration is given here to the fact that the energy deposition of the tissue is frequency-dependent.

Corresponding matching of emitted radiation, i.e. the frequency and field amplitude and also the magnetic field direction, to the implant can be implemented on part of the implant in particular by virtue of the design of the particle properties, e.g. their size, their crystallinity, their magnetic behaviour, in particular their magnetic relaxation, their stabilizing sheath, which has an effect on the homogeneous distribution of small agglomerates or no agglomerates in the polymer. The design of the particles also provides for an arrangement of individual particles in the polymer in the form of a chain or as an agglomerate, which has the amplified reaction to the applied magnetic field as a consequence.

As regards further technical features and advantages of the arrangement, reference is made to the description of the implant, to the figures and the description of the figures, and vice versa.

The invention is explained by way of example below with reference to the attached drawings, wherein the features presented below can in each case represent an aspect of the invention, both on their own and in combination, and wherein the invention is not restricted to the following drawing, the following description and the following exemplary embodiment.

In the figures:

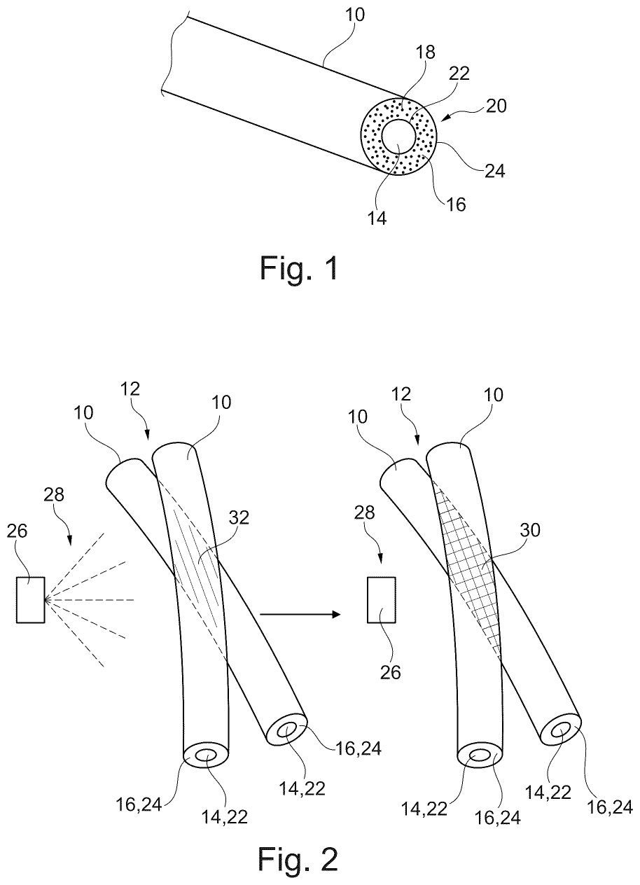

FIG. 1 shows a schematic view of a filament for an implant according to the present invention; and

FIG. 2 shows the mode of action of an implant according to the invention.

FIG. 1 shows a schematic view of a filament 10 for an implant 12 according to the present invention. The implant 12 serves in particular for implantation in a body, in particular in a hollow organ of a body. Moreover, the implant 12 can also be introduced into a vessel of a body.

The implant 12 is constructed from the filament 10, for instance in a braided structure. The filament 10 comprises at least one matrix material 14, 16, in which a magnetically heatable, in particular superparamagnetic, filler 18 is arranged and wherein at least the second matrix material 16 comprises a polymer. Further, FIG. 1 shows that the filament 10 has a cross section with a core-sheath structure 20.

It is evident that the core 22 has a thread-shaped configuration, precisely being configured as a fibre or filament, and the sheath 24 has a tubular structure and at least partially envelops the core 22.

In detail, in the implant 12 described here, provision is made for the core 22 to comprise a first in particular polymer matrix material 14 having a first melting point and for the sheath 24 to comprise a second polymer matrix material 16 having a second melting point, wherein the magnetically heatable filler 18 is present at least in the second matrix material 16. The second melting point is lower than the first melting point, in particular wherein the melting point difference between the first melting point and the second melting point is in a range of ≥5° C. and wherein the second melting point is in a range from ≥45° C. in particular to ≤100° C. This yields improved applicability of the implant 12.

It is also evident that the filler 18 is loaded more in the sheath 24 than in the core 22. In particular, the core 22 is free from the filler 18. Further, the core-sheath structure 20 or the filament 10 is embodied as a two-layer structure in particular.

To produce the implant 12, the filler 18, for example the nanoferrites to be used, is synthesized and compounded together with the polymer on a twin-screw extruder in order to form a spinnable compound. Subsequently, this compound is spun to form inductively heatable fibres using the melt spinning process. To create the core-sheath structure 20, coextrusion of core material and sheath material is implemented, in particular. The implant 12, such as in particular the stent, is displaced via a catheter system to the appropriate position in the body or in the hollow organ or vessel, and is subsequently expanded by means of self-expansion or balloon dilation in order to exert a certain amount of contact pressure. This profits from good flexibility of the implant 12 during the introduction.

Following an introduction of the implant 12 into a hollow organ or a vessel of a body, the implant 12 can be processed by means of a radiation source 26 for emitting electromagnetic radiation, as described below.

Upon excitation in an electromagnetic field, the filler 18 converts the absorbed energy from the field into heat, and outputs the latter to the surroundings. For instance, this is rendered possible by the use of precisely the radiation source 26 which emits electromagnetic radiation in such a way that the filler 18 is heated to a suitable temperature in order to melt the second matrix material 16. Together with the implant 12, the radiation source 26 can form a connected or matched arrangement 28.

In the case of a described implant 12, the stability can be improved postoperatively, i.e. after the introduction into a hollow organ of a body. In detail, the radial strength can be attained by way of forming an integral bond and thus forming crossing points 30. As indicated above, this is realized by virtue of the sheath 24 being heated above the melting point of the second matrix material 16, and hence previously loose contact points 32 forming crossing points 30 by melting and curing of the second matrix material 16. It should be mentioned that, within the scope of the present invention, the terms crossing points 30 and contact points 32 should be understood broadly and corresponding areas should be comprised thereby.

Forming the crossing points 30 can be realized at a time at which the implant 12 no longer needs to be compressible. For example, it is initially loaded into a catheter without connected crossing points 30 and brought to the position to be treated in minimally invasive fashion.

There, the implant 12 is expanded, for example by means of self-expansion or balloon dilation, and the contact points 32 of the filament 10 are integrally bonded with the formation of crossing points 30 only after the expansion, as shown in FIG. 2.

According to the invention, this thus results in a particularly advantageous combination of problem-free minimally invasive introducibility of the implant 12 as a result of great flexibility and a subsequent radial strength increase with the formation of crossing points 30 in the hollow organ.

### REFERENCE SIGNS

10 Filament
12 Implant
14 First matrix material
16 Second matrix material
18 Filler
20 Core-sheath structure
22 Core
24 Sheath
26 Radiation source
28 Arrangement
30 Crossing points
32 Contact point

1. Implant for implanting in a body, in particular in a hollow organ or a vessel of a body, wherein the implant is constructed from a filament containing at least one polymer matrix material in which a magnetically heatable filler is arranged, wherein the filament has a cross section with a core-sheath structure, characterized in that the core comprises a first, in particular polymer, matrix material having a first melting point and in that the sheath comprises a second, polymer matrix material having a second melting point, wherein the magnetically heatable filler is present at least in the second matrix material, wherein the second melting point is lower than the first melting point, wherein the second melting point is in a range from ≥45° C. in particular to ≤100° C.

2. Implant according to claim 1, characterized in that the second melting point is in a range from ≥45° C. to ≤65° C.

3. Implant according to claim 1, characterized in that the second melting point is in a range from >65° C. to ≤100° C.

4. Implant according to claim 1, characterized in that a melting point difference between the first melting point and the second melting point is in a range of ≥5° C., for example ≥20° C.

5. Implant according to claim 1, characterized in that the first matrix material and the second matrix material are biodegradable.

6. Implant according to claim 1, characterized in that the first matrix material is selected from the group consisting of polylactides, polylactide-co-glycolides, polyglycolides, poly-hydroxybutyrates, polydioxanone.

7. Implant according to claim 1, characterized in that the second matrix material is selected from the group consisting of polycaprolactone and a copolymer of polyglycolic acid and E-caprolactone.

8. Implant according to claim 1, characterized in that an active ingredient is present in the second matrix material.

9. Implant according to claim 1, characterized in that the core is free from the filler.

10. Implant according to claim 1, characterized in that the core-sheath structure is created by a coextrusion process.

11. Implant according to claim 1, characterized in that the magnetically heatable filler comprises a ferromagnetic or ferrimagnetic, in particular superparamagnetic, filler.

12. Implant according to claim 1, characterized in that the filament has a braided structure.

13. Arrangement of a radiation source for emitting electromagnetic radiation and an implant, wherein the implant contains a magnetically heatable filler, characterized in that the implant is configured according to claim 1.

14. Arrangement according to claim 13, characterized in that the implant and the radiation source are matched to one another in such a way that the implant is heatable, at least in the sheath, to a temperature in a range from ≥45° C. in particular to ≤100° C. by electromagnetic radiation emitted by the radiation source.

15. Arrangement according to claim 13, characterized in that the radiation source is set up to emit radiation at a frequency in a range from 10 kHz to 1 MHz in a field amplitude range of 1 kA/m to 100 kA/m.
